(19) United States
(12) Patent Application Publication
(10) Pub. No.: US 2011/0272599 A1
(43) Pub. Date: Nov. 10, 2011

(54) DEVICE FOR IRRADIATING MEDIA WITH UV LIGHT

(76) Inventors: Michael Wisskirchen, Albershausen (DE); Peter Voss, Esslingen (DE); Ferdinand Mayer, Oberboihingen (DE); Bjoern Blossey, Uhingen (DE); Werner Valder, Niedeggen/Abenden (DE)

(21) Appl. No.: 13/070,595

(22) Filed: Mar. 24, 2011

(30) Foreign Application Priority Data
May 5, 2010 (DE) 10 2010 019 335.8

(51) Int. Cl. G21K 5/00; A61L 2/10

(52) U.S. Cl. 250/492.1

(57) ABSTRACT

A device for irradiating media with UV light has a housing which contains at least one tubular body composed of UV-permeable material and through which medium flows, and at least one UV light source disposed at a distance from the tubular body and approximately parallel thereto, and to which a reflector is assigned. A reflector is likewise assigned to the at least one tubular body, which is disposed opposite the reflector of the UV light source and is open toward same.

### CROSS-REFERENCE TO RELATED APPLICATION

The invention described and claimed hereinbelow is also described in German Patent Application DE 10 2010 019 335.8 filed on May 5, 2010. This German Patent Application, whose subject matter is incorporated here by reference, provides the basis for a claim of priority of invention under 35 U.S.C. 119(a)-(d).

### BACKGROUND OF THE INVENTION

The invention relates to a device for irradiating media with UV light, comprising a housing which contains at least one tubular body composed of UV-permeable material and through which medium flows, and at least one UV light source disposed at a distance from the tubular body and approximately parallel thereto, and to which a reflector is assigned.

Devices of this type are used in particular to sterilize liquids, and are used to particular advantage in aquaristics, for instance, to sterilize aquarium water.

In the case of a known device of the type referred to initially, a centrally disposed tubular body for the medium flow is provided, onto the outside of which at least two axially parallel UV light sources comprising reflectors are mounted. The light sources are in the form of flat UV emitters which have an elongated, flat oval cross section having wide and narrow sides. Each of the narrow sides of the UV light sources is directed by way of the central axis thereof toward the center point of the tubular body cross section, and the radiation emitted from the broad sides of the flat UV emitter is deflected toward the centrally disposed tubular body using the reflectors which are assigned to the UV light sources. It has been shown that, in the case of such a device, the sterilization effect that can be attained for medium conducted therethrough and which is to be sterilized, e.g. aquarium water in particular, is inadequate. This is due to the type of UV light source and the associated reflectors installed on the central tubular body. Moreover, the device is bulky and takes up a relatively large amount of space. It basically cannot be designed with a compact and pleasing shape.

### SUMMARY OF THE INVENTION

The problem addressed by the invention is that of creating a device of the type referred to initially, which has a compact, space-saving, and simple design, and improves sterilization.

The problem is solved in the case of a device of the initially noted type according to the invention by assigning a reflector to the at least one tubular body, which is disposed opposite the reflector of the UV light source and is open toward same. The device according to the invention therefore comprises a reflector which is assigned to the UV light source, and a reflector which is assigned to the at least one tubular body. It has been shown that the beam path that is attained results in a considerable improvement of the sterilization of medium conducted therethrough. The device operates reliably, and is compact and economical. It requires less space.

According to an advantageous embodiment, the reflector of the at least one UV light source is shaped approximately as the arc of a circle, and is approximately semi-circular in particular. Likewise, the reflector of the tubular body can also be shaped approximately as the arc of a circle, and can be approximately semi-circular in particular. Advantageously, the two reflectors can extend along a common circular arc, in particular a circle, it being possible for the two reflectors to be combined to form a reflector circle. Each reflector can be disposed on the inner side of the housing, or can be present there in the form of a mirror coating, for example. It can be advantageous for the reflector circle to be formed of an approximately cylindrical tube having a reflecting surface on the inner side, and for this tube to be disposed in the interior of the housing between housing covers situated on both sides.

According to a further advantageous embodiment, the tubular body is disposed by way of the longitudinal central axis thereof approximately at the focal point, in particular on the focal point axis—which extends approximately parallel to the longitudinal central axis—of the reflector assigned thereto. In that particular case it is advantageous for the focal point, in particular the focal point axis, of the reflector of the tubular body and/or the reflector of the UV light source to lie within a diametral plane and approximately at one half the radius of the circular arc, in particular of the circle. It has been shown that this results in extraordinarily good, direct irradiation of the medium being conducted through the tubular body, and therefore sterilization of same.

It can be advantageous for the UV light source to comprise several, e.g. two, rods which extend next to one another approximately in parallel and have a common base which is used to hold said rods on a cover piece of the housing such that the cover piece can be removed together with the UV light source as a unit. Access to the UV light source, for replacement thereof, for instance, is thereby simplified and the design is compact.

It can be advantageous for the two rods of the UV light source to extend by way of the longitudinal central axes thereof within a rod longitudinal plane common to both, and for the focal point, in particular the focal point axis, of the associated reflector to lie within the rod longitudinal plane. In the other case, in which the UV light source comprises only one rod, the focal point, in particular the focal point axis, of the associated reflector is preferably located on the longitudinal central axis of said rod.

In the case of a UV light source formed of several, e.g. two, rods, the configuration can be selected such that the rods are disposed in the housing such that the rod longitudinal plane common to both extends radially and diametrically opposite the tubular body. Given this radial configuration it can be advantageous for the focal point, in particular the focal point axis, of the reflector of the tubular body to extend within the particular diametral plane within which the rod longitudinal plane extends. In that case, the focal point, in particular the focal point axis which extends approximately parallel to the rod longitudinal plane, of the associated reflector can be located at a distance from the circle center that corresponds approximately to one half of the radius.

According to another advantageous embodiment, the UV light source is rotated by 90° relative thereto, such that the rod longitudinal plane common to the rods lies on a circular secant which extends at a right angle to a diametral plane. It can be advantageous in this case for this circular secant to be situated a distance away from the circle center which corresponds approximately to one half the radius, and to be diametrically opposite the tubular body. The focal point, in particular the focal point axis, of the reflector of the UV light source can extend, advantageously, within a diametral plane situated approximately at a right angle to the circular secant, at a distance of approximately one half the radius from the center of the circle. It can be advantageous for the focal point, in particular the focal point axis, of the reflector of the tubular body to also lie within the diametral plane and approximately at one half the radius from the center of the circle.

According to an advantageous embodiment, the two rods of the UV light source, and the tubular body form a triangle. The tubular body can be disposed in the housing such that the longitudinal central axis thereof extends approximately parallel to the longitudinal extension of the UV light source and to the longitudinal central axis of the housing. The tubular body can be connected in a leak-proof manner at one end to a connector for the inflow, and at the opposite end to a connector for the outflow of the medium to be sterilized, wherein the connectors align with the longitudinal central axis of the tubular body. Due to this coaxial configuration of the connectors and the tubular body, the medium to be sterilized flows horizontially in a longitudinal direction. As a result, any pressure loss that may occur as the medium is conducted through is very low, and so any drop in the flow rate is likewise very low.

It can be advantageous for each connector to be approximately tubular and to be placed, inserted in particular, into the assigned tube ends of the tubular body, being sealed relative thereto. This results in a particularly simple and compact design.

Each connector can extend outwardly through an opening in a respective end-face housing cover, being sealed relative thereto. One of the housing covers can contain a passage for the UV light source with base, and an external sealing surface, against which the cover piece comprising the base with UV light source rests by way of a seal. This cover piece can be held on the housing cover in an easily detachable manner, using spring clips, for example. In some cases, it can simply rest thereon.

According to a further advantageous embodiment, the cover piece comprising the base with the UV light source, and the housing piece are covered by a detachable end cap, through which one of the connectors for inflow and outflow, and the electrical power supply for the UV light source are routed. This results in an economical design and a compact, attractive shape. The preconditions are created for easy detachment of the cover piece comprising the base with the UV light source without negatively affecting the seal integrity of the system.

The novel features which are considered as characteristic for the present invention are set forth in particular in the appended claims. The invention itself, however, both as to its construction and its method of operation, together with additional objects and advantages thereof, will be best understood from the following description of specific embodiments when read in connection with the accompanying drawings.

### BRIEF DESCRIPTION OF THE DRAWINGS

FIG. 1 shows a schematic perspective view of a device for irradiating media with UV light in accordance with the present invention,

FIG. 2 shows a schematic axial longitudinal view of the device in FIG. 1 in accordance with the present invention,

FIG. 3 shows a perspective exploded diagram of the device in FIG. 1 in accordance with the present invention,

FIG. 4 shows a schematic vertical view of the device in FIG. 1 in accordance with the present invention, on a larger scale.

### DESCRIPTION OF THE PREFERRED EMBODIMENTS

The drawings show schematic representations of a device 10 designed to irradiate media with UV light, in particular for the sterilization of liquids. Devices 10 of this type are particularly advantageous for use in aquaristics, for example.

Device 10 comprises an approximately cylindrical housing 11 which is closed at both ends using housing covers 12 and 13, respectively. Housing covers 12, 13 are approximately circular and comprise approximately rectilinear base parts 14 and 15 which extend beyond housing 11 and are used for placement on suitable surfaces. Extending away from one housing cover 13 is a strip-shaped tab 16 having hook 17 on the end, which is used for handling and for hanging on an aquarium wall, for example. Housing 11 contains at least one tubular body 18, through which medium flows, and which is composed of UV-permeable material such as plastic. Housing 11 also contains at least one UV light source 28 which is disposed at a distance from tubular body 18 and approximately parallel thereto.

Tubular body 18 is disposed in housing 11 such that longitudinal central axis 19 of tubular body 18 extends approximately parallel to the longitudinal extension of UV light source 28 and to longitudinal central axis 20 of housing 11. Tubular body 18 is connected in a leak-proof manner at each end to a connector 21 and 22, one of which is used for the inflow of the medium into tubular body 18, and the other of which is used for the outflow of the medium out of tubular body 18. Connectors 21, 22 align with longitudinal central axis 19. Each connector 21, 22 is approximately tubular in shape and is placed, e.g. inserted, into the respective assigned end of tubular body 18 and is sealed relative thereto. Two sealing rings 23, 24 are used for sealing, for example. Connectors 21, 22 extend outwardly through an opening 25, 26 in end-face housing cover 12, 13, respectively, and are sealed relative thereto. A sealing ring 29, 30 on connector 21, 22, respectively, is used for sealing.

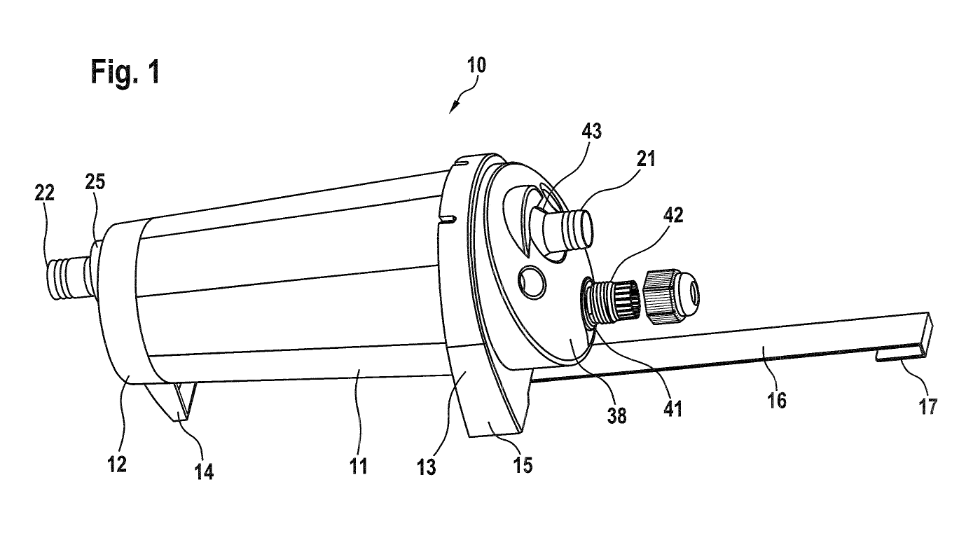

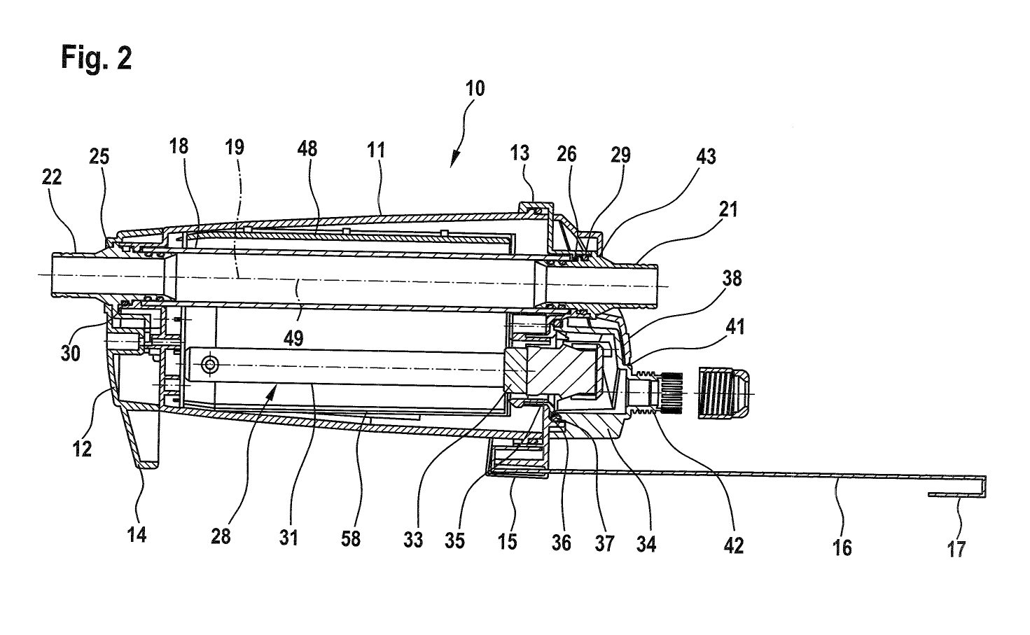

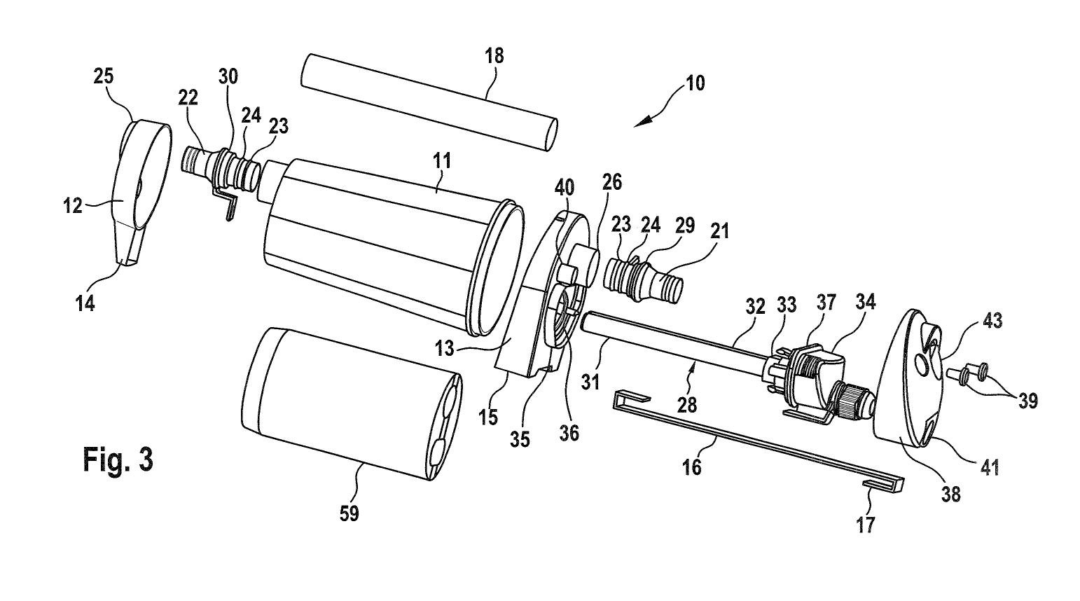

UV light source 28 can comprise a rod or, as provided in the embodiment shown, several, e.g. two, rods 31, 32 which extend next to one another approximately in parallel and have a common base 33. Base 33 holds rods 31, 32 on a cover piece 34 of housing 11 such that cover piece 34 can be detached together with UV light source 28 as a unit without affecting the seal integrity of the system. One of the housing covers, namely housing cover 13 in FIGS. 1 to 3, comprises a passage 35 for UV light source 28 with base 33 and an outer sealing surface 36, against which cover piece 34 comprising base 33 with UV light source 28 rests by way of a circumferential seal 37. Cover piece 34 is approximately triangular in shape.

Corresponding thereto, circumferential seal 37 likewise has an approximately triangular shape. Cover piece 34 comprising base 33 with UV light source 28, and housing cover 13 are covered by a detachable end cap 38 which rests on the end-face outer side of housing cover 13 and accommodates cover piece 34 in the interior. End cap 38 is attached to housing cover 13 using screws 39 which extend through holes in end cap 38 to assigned threaded sockets 40 of housing cover 13, into which they are screwed. End cap 38 contains a passage 41 for a cable gland 42 which extends outwardly through passage 41 and is used to accommodate the cable of the electrical power supply to UV light source 28. End cap 38 also has a through hole 43, through which a connector 21 for the inflow or outflow of the medium extends outwardly.

To disassemble device 10, e.g. for repair and in particular to replace UV light source 28, one simply loosens screws 39 so that end cap 38 can be removed. Cover piece 34 comprising UV light source 28 with base 33 can then be removed. The seal of the route through which the medium is conducted from one connector 21 by way of tubular body 18 to the other connector 22 remains unaffected, which is also true for the seal of respective connectors 21, 22 relative to respective housing cover 12, 13. For assembly, cover piece 34 with seal 37 is simply placed on housing cover 31, and end cap 38 is attached to cover piece 13 using screws 39 and threaded sockets 40 which accommodate same. It is recommended that seal 37 in cover piece 34 be replaced at that time. It is clear that the medium flows through connectors 21, 22 and coaxial tubular body 18 in a rectilinear manner. The flow is therefore horizontal. As a result, any loss of pressure in the medium is extraordinarly minor, and so any drop in flow rate is correspondingly minor.

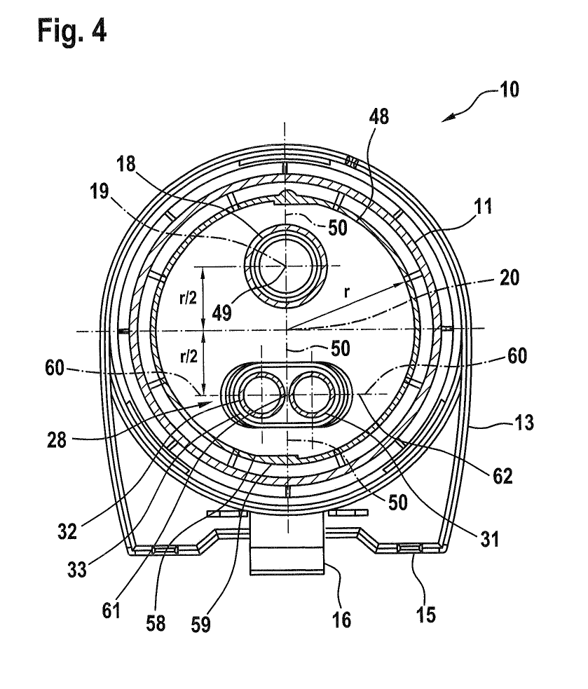

As shown in FIG. 4 in particular, the two rods 31, 32 of UV light source 28, and tubular body 18 are arranged in a triangle. A reflector 48 in housing 11 is assigned to the at least one tubular body 18. A reflector 58 is likewise assigned to UV light source 28. Reflectors 48, 58 are situated opposite one another and are open toward one another. They are both shaped approximately as the arc of a circle, and are preferably approximately semi-circular. They lie on a common circular arc, in particular a circle. Advantageously, reflector 48 of tubular body 18 and reflector 58 of the UV light source combine to form a reflector circle, as shown in FIG. 4 in particular. In the embodiment shown, the two approximately semi-circular reflectors 48, 58 are combined to form a tube 59. Tube 59 is accommodated and fastened in the interior of housing 11. It is provided with a reflecting surface, e.g. a mirror coating. According to another embodiment which is not shown, housing 11 is equipped instead on the inner side directly with each reflector 48, 58, e.g. to form a respective reflector having a mirror coating.

Tubular body 18 is disposed by way of longitudinal central axis 19 thereof approximately at the focal point of associated reflector 48, in particular on the focal point axis which extends approximately parallel to longitudinal central axis 19. This focal point and the focal point axis are labelled with reference numeral 49 in the drawings. It can be particularly advantageous for focal point 49 and the focal point axis of reflector 48 of tubular body 18 to be situated at approximately one half the radius of the circular arc, in particular of the circle formed by the two reflectors 48, 58. Longitudinal central axes 19 and 20 lie in a diametral plane labelled with reference numeral 50. Focal point 49, in particular the focal point axis, lies within diametral plane 50. If device 10 is equipped with only one rod as UV light source 28, it is expedient for the longitudinal central axis of this one rod to lie within diametral plane 50, and therefore focal point 61 and the focal point axis of reflector 58 are located approximately at one half of the radius of the circle formed by the two reflectors 48, 58. Focal points 49, 61 of the two reflectors 48, 58 are therefore situated opposite one another within diametral plane 50 and are each situated at a distance from circle center 20 that corresponds to approximately one half of the radius of the circle.

If UV light source 28 comprises two rods 31, 32, as shown in the embodiment, they are advantageously situated such that the longitudinal central axes of rods 31, 32 extend within a rod longitudinal plane 60 common to both. In that particular case it is advantageous for focal point 61 and the focal point axis of reflector 58 to lie within rod longitudinal plane 60. This applies for the case shown in FIG. 4, in which rods 31, 32 are disposed in housing 11 such that rod longitudinal plane 60 lies on a circular secant 62 that is oriented approximately at a right angle to diametral plane 50. It can be advantageous in this case for circular secant 62 to be situated at a distance from circle center 20 that corresponds approximately to one half of the radius, and to be diametrically opposite tubular body 18. Focal point 61, in particular the focal point axis, of reflector 58 of UV light source 28 is therefore located within diametral plane 50 at a distance from circle center 20 that corresponds approximately to one half of the radius, diametral plane 50 being oriented approximately at a right angle to circular secant 62. Focal point 49, in particular the focal point axis, of the other reflector 48 of tubular body 18 lies within diametral plane 50 and likewise at approximately one half the radius from circle center 20. It has been shown that, given such a configuration, a beam path starting from UV light source 28 can be attained that results in direct irradiation of the medium conducted in tubular body 18, thereby greatly improving sterilization.

According to another embodiment which is not shown, rods 31, 32 are disposed in housing 11 such that rod longitudinal plane 60 extends radially and diametrically opposite tubular body 18. In such a case, focal point 49, in particular the focal point axis, of reflector 48 of tubular body 18 lies within particular diametral plane 50 within which rod longitudinal plane 60 also extends. In that particular case, the half-way point of rod longitudinal plane 60 can be located at a distance from circle center 20 that corresponds to approximately one half of the radius, and can be situated approximately at focal point 61, in particular on the focal point axis—which extends approximately parallel to rod longitudinal plane 60—of reflector 58. In that particular case, rod longitudinal plane 60 is therefore rotated by 90° relative to the depiction shown in FIG. 4 and coincides with diametral plane 50. After said rotation, focal point 61, in particular the focal point axis, remains at the same point at a distance from circle center 20 that corresponds to approximately one half the radius of the circle.

It is clear that device 10 is simple, is composed of only a few components, and can therefore be developed at low cost. The handling thereof is particularly simple. Device 10 is highly effective. Sterilization results are improved by irradiating the medium that passes through tubular body 18 as directly as possible. Since the medium passes through connectors 21, 22 and tubular body 18 in a linear manner, any pressure losses are reduced substantially, and the greatest possible flow rate is attained.

It will be understood that each of the elements described above, or two or more together, may also find a useful application in other types of constructions differing from the types described above.

While the invention has been illustrated and described as embodied in a device for irradiating media with UV light, it is not intended to be limited to the details shown, since various modifications and structural changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed as new and desired to be protected by Letters Patent is set forth in the appended claims:

1. A device for irradiating media with UV light, comprising a housing which contains at least one tubular body composed of UV-permeable material and through which medium flows and at least one UV light source disposed at a distance from the tubular body and substantially parallel thereto; a first reflector assigned to the at least one UV light source; and a second reflector which is assigned to the at least one tubular body, is situated opposite the first reflector of the UV light source, and is open toward the first reflector.

2. The device as defined in claim 1, wherein the first reflector of the at least one UV light source is shaped substantially as an arc of a circle.

3. The device as defined in claim 2, wherein the first reflector is substantially circular.

4. The device as defined in claim 1, wherein the second reflector of the tubular body is shaped substantially as an arc of a circle.

5. The device as defined in claim 1, wherein the second reflector is substantially semi-circular.

6. The device as defined in claim 1, wherein the first reflector of the at least one UV light source and the second reflector of the tubular body extend along a common circular arc.

7. The device as defined in claim 6, wherein the first reflector of the at least one UV light source and the second reflector of the tubular body extend along a circle.

8. The device as defined in claim 1, wherein the second reflector of the tubular body and the first reflector of the at least one UV light source combine to form a reflector circle.

9. The device as defined in claim 1, wherein each of the first and second reflectors is disposed in a manner selected from the group consisting of on an inner side of the housing and in a form of a mirror coating.

10. The device as defined in claim 1, wherein the tubular body is disposed by a longitudinal central axis thereof substantially at a focal point of the second reflector.

11. The device as defined in claim 10, wherein the tubular body is disposed on a focal point axis which extends approximately parallel to the longitudinal central axis.

12. The device as defined in claim 10, wherein the focal point of a reflector selected from the group consisting of the second reflector of the tubular body, the first reflector of the UV light source, and both is located within a diametral plane and substantially at one half a radius of a circular arc.

13. The device as defined in claim 12, wherein the focal point of the reflector selected from the group consisting of the second reflector of the tubular body, the first reflector of the UV light source, and both is located substantially at one half a radius of a circle.

14. The device as defined in claim 1, wherein the UV light source comprises several rods which extend next to one another substantially in parallel, and have a common base, with which they are held on a cover piece of the housing such that the cover piece is removable together with the UV light source as a unit.

15. The device as defined in claim 14, wherein the rods include two rods which extend by longitudinal central axes thereof within a rod longitudinal plane common to both, and a focal point of the first reflector lies within the rod longitudinal plane.

16. The device as defined in claim 14, wherein the rods include two rods which extend by a longitudinal central axes thereof within a rod longitudinal plane common to both, and a focal point of the first reflector lies within the rod longitudinal plane, wherein a focal point axis of the first reflector lies within the rod longitudinal plane.

17. The device as defined in claim 15, wherein the rods are disposed in the housing such that the rod longitudinal plane extends radially and diametrically opposite the tubular body.

18. The device as defined in claim 17, wherein a focal point of the second reflector of the tubular body extends within a diametral plane within which the rod longitudinal plane extends.

19. The device as defined in claim 18, wherein a focal point axis of the second reflector of the tubular body extends within the diametral plane within which the rod longitudinal plane extends

20. The device as defined in claim 18, wherein a half-way point of the rod longitudinal plane is located at a distance from a circle center that corresponds substantially to one half of a radius, and is situated substantially in a focal point which extends substantially parallel to the rod longitudinal plane of the first reflector.

21. The device as defined in claim 20, wherein the half-way point is located on a focal point axis which extends substantially parallel to the rod longitudinal plane of the first reflector.

22. The device as defined in claim 14, wherein the rods are disposed in the housing such that a rod longitudinal plane lies on a circular secant which extends at a right angle to a diametral plane, is located at a distance from a circle center substantially equal to one half a radius, and is diametrically opposite the tubular body.

23. The device as defined in claim 22, wherein a focal point of the first reflector of the UV light source is located within a diametral plane—which is oriented substantially at a right angle to a circular secant—at a distance from a circle center that corresponds substantially to one half a radius.

24. The device as defined in claim 23, wherein a focal point axis of the first reflector of the UV light source is located within a diametral plane—which is oriented substantially at a right angle to a circular secant—at a distance from a circle center that corresponds substantially to one half a radius.

25. The device as defined in claim 1, wherein a focal point of the second reflector of the tubular body lies within a diametral plane and at substantially one half the radius from a circle center.

26. The device as defined in claim 25, wherein a focal point axis of the second reflector of the tubular body lies within the diametral plane and at substantially one half the radius from the circle center.

27. The device as defined in claim 15, wherein the two rods of the UV light source, and the tubular body are arranged in a triangle.

28. The device as defined in claim 1, wherein the tubular body is disposed in the housing such that a longitudinal central axis of the tubular body extends substantially parallel to a longitudinal extension of the UV light source and a longitudinal central axis of the housing, and the tubular body is connected in a leak-proof manner at one end to a connector for inflow, and at an opposite end to a connector for outflow, which align with the longitudinal central axis of the tubular body.

29. The device as defined in claim 28, wherein each of the connectors is substantially tubular and is placed in a leak-proof manner.

30. The device as defined in claim 29, wherein each of the connectors is inserted in assigned tube ends of the tubular body in the leak-proof manner.

31. The device as defined in claim 28, wherein each of the connectors extends outwardly through an opening in a respective end-face housing cover, and is sealed relative thereto.

32. The device as defined in claim 31, wherein one of the housing covers comprises a passage for the UV light source with a base, and an outer sealing surface, against which a cover piece comprising the base with the UV light source rests by a seal.

33. The device as defined in claim 32, wherein the cover piece comprising the base with the UV light source, and the housing cover are covered by a detachable end cap, through which one of connectors for an inflow or an outflow, and an electrical power supply for the UV light source are routed.

34. The device as defined in claim 8, wherein the reflector circle is formed of a substantially cylindrical tube which has a reflecting surface on an inner side and is disposed in an interior of the housing between two housing covers.
